(19) United States
(12) Patent Application Publication
(10) Pub. No.: US 2023/0285558 A1
(43) Pub. Date: Sep. 14, 2023

(54) ENGINEERED T CELLS AND TUMOR-INFILTRATING LYMPHOCYTES TO INCREASE TUMOR KILLING

(71) Applicant: Board of Regents, The University of Texas System, Austin, TX (US)

(72) Inventors: Patrick HWU, Houston, TX (US); Barbara M. NASSIF RAUSSEO, Houston, TX (US)

(73) Assignee: Board of Regents, The University of Texas System, Austin, TX (US)

(21) Appl. No.: 18/006,652

(22) PCT Filed: Jul. 23, 2021
(86) PCT No.: PCT/US2021/070943

§ 371 Date: Jan. 24, 2023

Related U.S. Application Data
(60) Provisional application No. 63/056,467, filed on Jul. 24, 2020.

(51) Int. Cl. A61K 39/00; C12N 5/0783; C12N 15/113; A61K 45/06; A61P 35/00

(52) U.S. Cl. CPC A61K 39/4631; C12N 5/0638; C12N 15/1138; A61K 45/06; A61P 35/00

(57) ABSTRACT

Embodiments of the disclosure encompass methods and compositions related to cell therapy treatment, including for cancer. In specific embodiments, the disclosure concerns adoptive cell therapy cancer treatment in which tumor-1 infiltrating lymphocytes and/or engineered T cells are modified to increase their efficacy as a cancer treatment. In specific cases, the cells are engineered for knock out of one or more genes, such as Signaling Threshold Regulating Transmembrane Adaptor 1 (SIT1), Bone Marrow Stromal Cell Antigen 2 (BST2), and/or programmed cell death protein 1 (PD-1).

This application claims priority to U.S. Provisional Patent Application Ser. No. 63/056,467, filed Jul. 24, 2020, which is incorporated by reference herein in its entirety.

### TECHNICAL FIELD

Embodiments of the disclosure include at least the fields of cell biology, molecular biology, immunology, cell therapy, and medicine.

### BACKGROUND

Cellular immunotherapy holds much promise for the treatment of cancer. However, certain cellular therapies have limited success because of immunosuppression in the tumor microenvironment. Thus, there is an unmet need for improved methods of cellular immunotherapy.

### BRIEF SUMMARY

The present disclosure concerns methods and compositions related to cell therapies for any purpose, including at least for treatment of cancer of any kind. The disclosure particularly concerns adoptive cell therapies for individuals with cancer, including methods and compositions that allow immunotherapies to work more effectively for killing cancer cells of any kind. The methods and compositions increase efficacy of the cells for the therapy in at least some cases by allowing them to overcome immunosuppression at the tumor microenvironment. In particular embodiments, the cells are modified to reduce expression completely or partially of multiple endogenous genes in the cells, and such modifications allow the cells of the therapy to kill cancer cells more effectively than if the cells were not so modified.

Particular embodiments of the disclosure concern modulation of particular immune cell surface proteins to enhance adoptive cell therapy, and in specific cases the immune cells are lymphoid cells, including at least T cells, tumor infiltrating lymphocytes, or B cells, for example. The methods and compositions relate to modulation of expression of multiple genes whose products are involved in suppression of the cells, in particular aspects. In specific embodiments, the modulation of expression concerns downregulation of expression of multiple genes, including in at least some cases to non-detectable levels, such as determined by any standard means in the art (at the RNA and/or protein level, for example). The modulation of expression of the genes in the cells may be for the explicit purpose of improving efficacy of the cells for therapy, including improving efficacy in the tumor microenvironment.

In particular embodiments, the present disclosure involves the knockout (or knockdown, in certain cases) of genes in particular lymphoid cells, such as tumor-infiltrating lymphocytes (TILs) or engineered T cells, for example, to improve the function of the cells within the tumor microenvironment. This disclosure provides adoptive cell therapy embodiments that allow therapeutic efficacy in individuals with cancer, including solid tumors. In particular embodiments, the genes that are knocked out or knocked down are Signaling Threshold Regulating Transmembrane Adaptor 1 (SIT1), Bone Marrow Stromal Cell Antigen 2 (BST2), and Programmed cell death protein 1 (PD-1). In particular embodiments, all three of SIT1, BST2, and PD-1 are knocked out or knocked down, although in alternative embodiments only one or two of SIT1, BST2, and PD-1 are knocked out or knocked down in the cells. Thus, in specific embodiments, the expression of SIT1, BST2, and PD-1 is altered through genetic modification that results in improved T cell resistance or TIL resistance to immunosuppression compared to T cells or TIL, respectively, that do not have deliberate modification of expression of these genes. The modification may be of any kind, including by CRISPR or RNA interference, for example.

Embodiments of the disclosure allow for improved T cell resistance to immunosuppression compared to T cells with un-altered expression of SIT1, BST2, and PD-1, including to overcome immunosuppression in the tumor microenvironment. Specific embodiments provide methods and compositions for eliminating SIT1, BST2, and PD-1 having a direct or indirect role in T cell suppression, thereby overcome T cell suppression.

In certain embodiments, the adoptive cells of the therapy are modified in ways other than downregulation of SIT1, BST2, and PD-1, such as having one or more engineered antigen receptors. For example, the immune cells may be modified to express one or more engineered receptors, including engineered antigen receptors, such as engineered T cell receptors (TCR) and/or chimeric antigen receptors (CAR). The specific antigen(s) to which the TCR and/or CAR is directed may be any kind of antigen, although in specific cases the antigen is expressed on the cancer cells of the individual receiving the adoptive cell therapy.

The adoptive cell therapy may be autologous or allogeneic (or a mixture thereof) with respect to a recipient individual. In certain embodiments, autologous or allogeneic T cells or TILs are harvested from human donors (the donor being self if the T cells are allogeneic or from another individual if the T cells are autologous) and cultured ex vivo. Autologous or allogeneic cells undergo gene editing ex vivo, such as by using CRISPR/Cas9 ribonucleoprotein complexes, or similar. The edited and expanded cells are then administered, such as by infusion, into individuals with cancer as an adoptive cell therapy.

Embodiments of the disclosure include compositions comprising at least one engineered immune cell, wherein the cell is engineered to have disruption in expression of the following endogenous genes: (a) Signaling Threshold Regulating Transmembrane Adaptor 1 (SIT1), (b) Bone Marrow Stromal Cell Antigen 2 (BST2), and (c) Programmed cell death protein 1 (PD-1). In specific embodiments, the immune cell is a lymphoid cell, such as a tumor-infiltrating lymphocyte (TIL), T cell, B cell, or a mixture thereof. The immune cell may particularly be a TIL or T cell. In specific embodiments, the cell is obtained from an individual in need of cell therapy, such as an individual with cancer. In some cases the cell is obtained from the cancer of the individual. The cells may be obtained from a repository. In specific embodiments, the composition comprises a population of the engineered immune cells. The cells may be autologous with respect to a recipient individual or allogeneic with respect to a recipient individual. Any immune cell may be a TIL or T cell that is positive for cluster of differentiation 8 (CD8). In some cases, when the cells are T cells or TILs, they have disruption in expression of the native T cell receptor.

In particular embodiments, disruption in expression comprises a knockout of SIT1, BST2, and PD-1. The disruption in expression may comprise a knockdown of SIT1, BST2, and PD-1. In some cases, the disruption in expression is a knockout of one or two of SIT1, BST2, and PD-1, and the disruption in expression is a knockdown of the respective remaining two or one of SIT1, BST2, and PD-1. The disruption in expression may be the result of CRISPR gene editing.

Any of the engineered cells of the disclosure may comprise a disruption in expression in one or more inhibitory molecules, such as cytotoxic T-lymphocyte-associated protein 4 (CTLA4), T-cell immunoglobulin mucin-3 (TIM-3), Lymphocyte Activating 3 (LAG3), or a combination thereof. The cells alternatively or additionally may comprise expression of one or more engineered receptors, such as engineered antigen receptor, including one or more chimeric antigen receptors and/or one or more engineered T cell receptors. Any composition may comprise a pharmaceutically acceptable carrier.

In embodiments of the disclosure, there is a method of producing any cell or cells of the disclosure, comprising the step of subjecting one or more immune cells to disruption of expression of the endogenous SIT1, BST2, and PD-1 genes in the immune cell or cells. In some cases, the disruption is performed by CRISPR gene editing. In some cases, prior to the subjecting step, the cells are expanded. The immune cell or cells may be obtained from blood, including at least peripheral blood. The immune cells may be obtained from a tumor.

Any method of the disclosure may comprise the step of expanding the cells. The expanding may occur prior to the disruption of expression. The expanding may occur subsequent to the disruption of expression. In any method, there may include modifying of the cells to express one or more engineered receptors. The modifying of the cells to express one or more engineered receptors may occur prior to the disruption of expression. The modifying of the cells to express one or more engineered receptors may occur subsequent to the disruption of expression.

In some embodiments, there is a method of improving efficacy of immune cell therapy, comprising the step of disrupting expression of SIT1, BST2, and PD-1 in the immune cells of the immune cell therapy. The disrupting of expression in the cells overcomes immunosuppression for the cells in the microenvironment of a tumor, in at least some cases.

Embodiments of the disclosure include methods of treating an individual for cancer, comprising the step of providing to the individual an effective amount of any one of the compositions of the disclosure. In some cases, the immune cells are obtained from the cancer of the individual. The method may be further defined as: (a) expanding immune cells obtained from the cancer of the individual; (b) disrupting expression of SIT1, BST2, and PD-1 in the cells to produce engineered cells; and (c) administering an effective amount of the engineered cells to the individual. In some cases, the method comprises the step of: (d) modifying the engineered cells to express one or more engineered receptors. The engineered receptors may be engineered antigen receptors, including at least one or more chimeric antigen receptors and/or one or more engineered T cell receptors. Any method of the disclosure may include the step of providing to the individual an effective amount of an additional cancer therapy, such as radiation therapy, surgery, chemotherapy, gene therapy, DNA therapy, viral therapy, RNA therapy, immunotherapy, bone marrow transplantation, nanotherapy, monoclonal antibody therapy, hormone therapy, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the invention are utilized, and the accompanying drawings (also “Figure” and “FIG.” herein), of which:

FIGS. 1A and 1B provide survival melanoma data in association with expression of SIT1. SIT1 expression is representative of immune cell infiltration due to SIT1 being selectively expressed in immune cells. (The Cancer Genome Atlas (TCGA); Marie-Cardine et al., 1999).

FIG. 2 demonstrates preliminary staining in murine T cells that indicates SIT1 expression increases after T cell activation, in at least specific embodiments.

FIG. 3 provides survival melanoma data in association with expression of BST2, similar to PD-1 and SIT-1 expression (TCGA).

FIG. 4 demonstrates BST2 and SIT1 expression having a significant correlation in CD8+ T cells from metastatic melanoma lesions from non-responders to anti-PD1 immunotherapy. This data was obtained from single-cell RNAseq from the anti-PD1 non-responders group from data generated by Sade-Feldman et al., 2018. The correlative coefficient shows positive correlation between BST2 and SIT1 expression. The log 2 (transcripts per million) values used for this analysis come from the pre-therapy cells. The cells with zero counts for both genes were removed.

FIG. 5 provides an example of an in vitro approach for modulation of the gene or genes in mouse CD8+ T cells.

FIG. 6 shows that in vitro CRISPR/Cas9 knockout of SIT1 resulted in significant killing compared to controls. This figure shows results from a co-culture assay of murine tumor cells and T cells (also known as a caspase assay). The cytotoxicity of the modified mouse T cells was assessed by measuring the caspase enzyme levels in the tumor cells that represent apoptosis. A Non-targeting control (NTC) was utilize for comparison, representing tumor killing by unmodified cells. A Non-electroporated controls was also utilized that did not receive any Cas9, tracrRNA, or crRNA.

FIG. 7 shows that in vitro CRISPR/Cas9 knockout of BST2 resulted in significant killing compared to controls. A caspase assay was utilized as in FIG. 6.

FIG. 8 provides an example of an in vitro approach for modulation of the gene or genes in human TILs.

FIG. 9 shows that an in vitro multiplex CRISPR/Cas9 knockout of the three genes resulted in significant killing compared to controls including knockout of single genes.

FIG. 10 provides one approach for studies in vivo assessment of knockout of the gene or genes in certain mice.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### I. Examples of Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The term “subject” as used herein, generally refers to an individual in need of cell therapy and may be used interchangeably with the term “individual” or “patient.” The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more cancers. The subject may be undergoing or having undergone cancer treatment. The subject may be asymptomatic. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants. A subject may or may not have a need for medical treatment; an individual may voluntarily or involuntarily be part of experimentation whether clinical or in support of basic science studies.

The phrase “therapeutically effective amount” as used herein means that amount of a compound, material, or composition comprising a compound of the present disclosure that is effective for producing some desired therapeutic effect, e.g., treating (i.e., preventing and/or ameliorating) cancer in a subject, at a reasonable benefit/risk ratio applicable to any medical treatment. In one embodiment, the therapeutically effective amount is enough to reduce or eliminate at least one symptom. In specific embodiments, the amount of T cells may be determined by consideration of the individual's age, weight, tumor load or size, extent of disease (e.g., metastasis), and/or any additional health condition. One of skill in the art recognizes that an amount may be considered therapeutically effective even if the cancer is not totally eradicated but improved partially. For example, the spread of the cancer may be halted or reduced, a side effect from the cancer may be partially reduced or completely eliminated, the life span of the subject may be increased, the subject may experience less pain, the quality of life of the subject may be improved, and so forth.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “mammal” is an appropriate subject for the method of the present disclosure. A mammal may be any member of the higher vertebrate class Mammalia, including humans. Examples of mammals are humans, cats, dogs, cows, mice, rats, horses, goats, sheep, and chimpanzees. Mammals may be referred to as “patients” or “subjects” or “individuals”.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination; examples include CRISPR. Examples of modifications include insertions, mutations, and/or deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In some cases, the term is referring to cells that have been modified by the hand of man to harbor or express one or more molecules that are not found in nature. With respect to heterologous antigen receptors, the term “engineered” as used herein refers to antigen receptors that are generated by the hand of man and are not found in nature and are not endogenous to the cell that expresses it. The receptors may be synthetically generated through standard recombination techniques, for example. Examples include T cell receptors, chimeric antigen receptors, chimeric cytokine receptors, and so forth.

T cell-based adoptive cell therapies have resulted in dramatic response rates (>90%) in hematologic malignancies, but response rates are substantially lower in solid tumors. This is thought to be due in large part to immunosuppression in the tumor microenvironment. The present disclosure provides a method to improve the therapeutic efficacy of adoptive cell therapy in solid tumors by knocking out genes that are involved in T cell suppression. This addresses the market need for improved immunotherapies for patients with solid tumors, in particular cases.

Embodiments of the disclosure encompass methods and compositions for cell therapy treatment, including adoptive cell therapy cancer treatment. The disclosure in particular provides methods and compositions to allow cell therapy to be more effective in a recipient individual, including more effective at killing cancer cells in the individual. In particular embodiments, the cell therapy is more effective in a tumor microenvironment at least because the cells of the cell therapy are engineered specifically to overcome immunosuppression in the tumor microenvironment.

In particular embodiments, immune cells, including lymphoid cells, are engineered to increase efficacy of killing of cancer cells, and in specific embodiments the immune cells are engineered and are tumor-infiltrating lymphocytes (TILs), T cells, or a mixture thereof. The engineering of the cells encompassed by the disclosure allows the cells to be more effective as adoptive cell therapy for cancer treatment than in the absence of the engineering. In specific embodiments, the disclosure provides methods and compositions to allow engineered TILs and/or engineered T cells to be more effective in tumor microenvironments compared to use of TILs and/or engineered T cells in the absence of the disclosed modification(s) of the cells. The cells of the adoptive cell therapy may be engineered in other ways as well, such as by the expression of one or more engineered antigen receptors and/or one or more heterologous cytokine genes (including integration of one or more heterologous cytokine genes). Examples of cytokines include but are not limited to IL-2, IL-15, IL-7, IL-21, or a combination thereof.

### II. Engineered Cells of the Disclosure

Cells of the disclosure are engineered by the hand of man to have one or more advantageous properties, including for properties that enhances their ability to be used as a therapy for an individual in need thereof. The cells have substantially different marked characteristics compared to any cells in nature, including the cells being synthetically modified to lack expression (including lack detectable expression) or have reduced expression of 1, 2, 3, or more endogenous genes in the cells, and in specific cases 3 or at least 3 endogenous genes in the cells. In specific cases, the cells may be considered to have suppression of expression of 1, 2, 3, or more (including at least 3, in some cases) endogenous genes in the cells, and this suppression is not the result of naturally-occurring mutations, rearrangements, deletions, inversions, and so forth. In particular embodiments, there is disruption of expression of 1, 2, 3, or more endogenous genes in the cells, and this disruption would not occur without the hand of man.

The disruption in expression of the cells in certain cases refers to knockout of one or more endogenous genes and/or knockdown of one or more endogenous genes. In specific embodiments, the cells are knocked out or knocked down in expression of 1, 2, 3, or more endogenous genes in the cells. Knockout as used herein means the gene has been completely silenced, and knockdown as used herein means the expression has been reduced but some gene product (RNA, or both RNA and protein) is still made. In some cases, the gene(s) has reduced expression to the extent that there is no detectable expression of the gene with the present detection methods in the art, including, for example, any standard method(s) in the art. Such detection may ascertain levels of RNA or protein or both, and examples include by northern and Western techniques, for example.

In particular embodiments, any of the genes that are disrupted in expression herein may be disrupted in expression for any reason, including because of modification at the level of transcription or translation of the gene product. The modification may affect the ability to transcribe the corresponding mRNA. The modification may be because of a deletion of part or all of the gene, including of any number of exons, for example.

SIT1

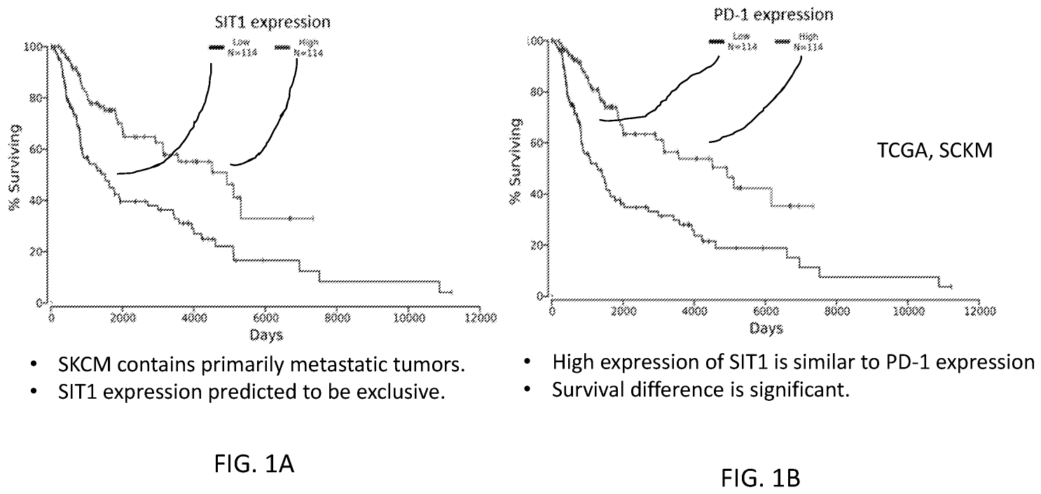

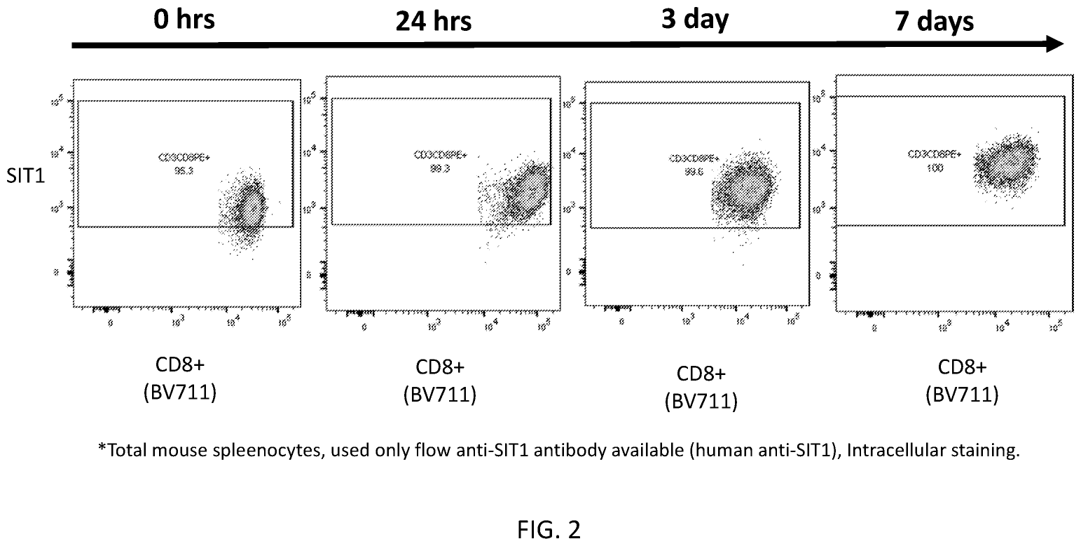

In particular embodiments, the cells have disrupted expression of the endogenous copy of Signaling Threshold Regulating Transmembrane Adaptor 1 (SIT1; GenBank® database Gene ID: 27240), which is also known as SIT, SIT-1, or SIT-R. As one example, a sequence of SIT 1 may be found at the National Center for Biotechnology Information's GenBank® database, Accession No. NM_014450. As one example, a protein sequence of SIT1 may be found at GenBank® Accession No. NP_055265, which is incorporated herein by reference. SIT1 is found in T cells, B cells, and plasma cells. It negatively regulates T-cell antigen receptor (TCR)-mediated signaling and is involved in positive selection of T-cells. Its structure includes a disulfide-linked homodimer and when phosphorylated, it interacts with PTPN11/SHP2, GRB2 and CSK. SIT1 expression is absent from normal skin, but according to The Cancer Genome Atlas (TCGA), melanoma has high expression of SIT1, which is representative of immune cell infiltration due to SIT1-exclusive expression in immune cells (Marie-Cardine et al., 1999). For melanoma, high expression of SIT1 versus low expression of SIT1 leads to a significant decrease in survival (FIGS. 1A and 1B; TCGA). In non-small cell lung cancer by single-cell sequencing, SIT1 expression is upregulated in tumor infiltrating lymphocytes in the tumor microenvironment and also in specifically exhausted CD8 T cells (Guo, X. et al. Nat. Med 24, 1628 (2018); from the produced data base; conclusion made from single-cell data at http://lung.cancer-pku.cn). Furthermore, SIT1 expression increases after T cell activation in mice (FIG. 2). It is known in the art that SIT1 phosphorylation increases after T cell receptor stimulation of human Jurkat cells (Pfrepper et al., 2001) and that SIT1-deficient murine T cells have enhanced activation and TCR signaling (murine) (Arndt et al., 2011).

BST2

In particular embodiments, the cells have disrupted expression of the endogenous copy of Bone Marrow Stromal Cell Antigen 2 (BST2; GenBank® database Gene ID:684), which is also referred to as BST2, CD317, and Tetherin. As one example, a nucleic acid sequence of BST2 may be found at GenBank® Accession No. NM_004335, which is incorporated herein by reference. As one example, a protein sequence of BST2 may be found at GenBank® Accession No. NP_004326, which is incorporated herein by reference. BST2 is a transmembrane glycoprotein well known for inhibiting the release of enveloped viruses from infected cells (Yi et al. Biochemical and Biophysical Research Communications, 509(2): 414-4202019). BST2 has also been shown to serve as the ligand for ILT7, a receptor on plasmacytoid dendritic cells that negatively regulates type I interferon expression (Bego et al. J Biol Chem. 294(27):10503-10518; 2019).

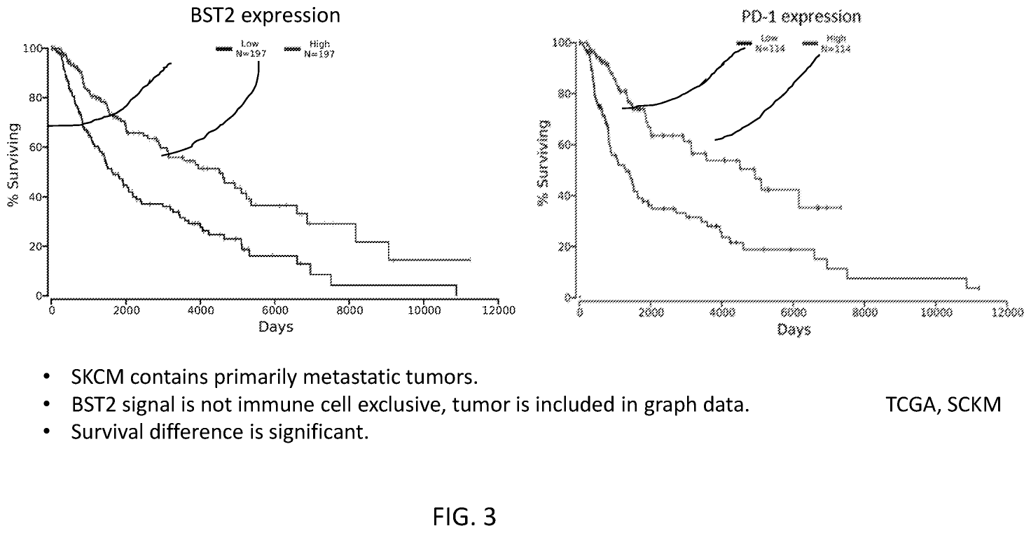

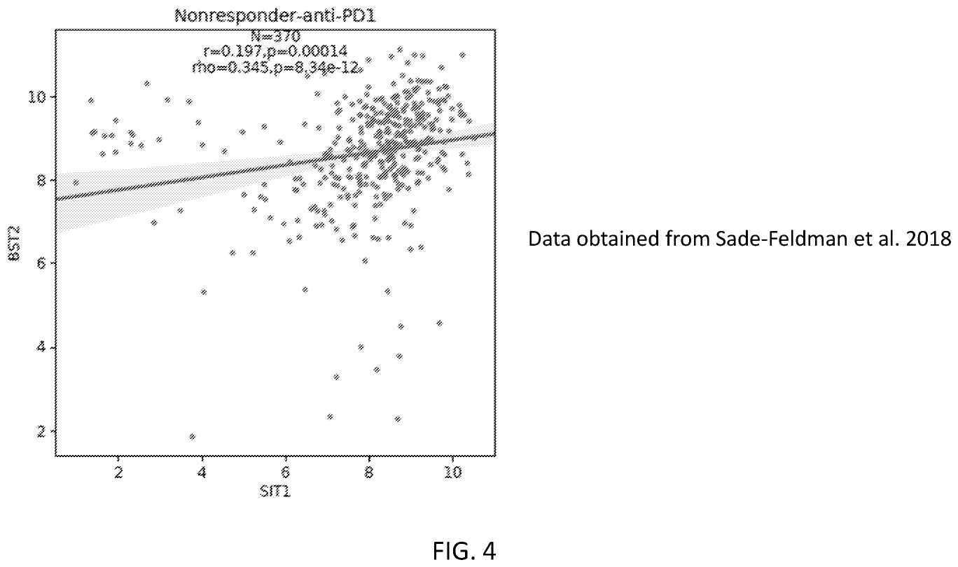

BST2 expression is low in normal skin, but TCGA melanoma data shows high expression of BST2 in melanoma (FIG. 3; TCGA). BST2 and SIT1 expression have a significant correlation with immunotherapy response (FIG. 4); correlation analysis shows significant positive correlation between BST2 and SIT1 expression in CD8+ T cells from patients that failed anti-PD1 therapy.

PD-1

In particular embodiments, the cells have disrupted expression of the endogenous copy of programmed cell death protein 1 (PD-1; GenBank® database Gene ID:5133), which is also known as PDCD1, CD279, PD1, SLEB2, hPD-1, hPD-1, hSLE1, or Programmed cell death 1. As one example, a sequence of PD-1 may be found at the National Center for Biotechnology Information's GenBank® database, Accession No. NM_005018. As one example, a protein sequence of PD-1 may be found at GenBank® Accession No. NP_0050009, which is incorporated herein by reference. PD-1 is a known inhibitor surface molecule on T cells and is involved in the role of immune suppressive signals from antigen presenting cells in the tumor microenvironment, leading to functionally exhausted T cells (Freeman and Sharpe, Nat Immunol., 2012, vol 13(2): pp. 113-115).

The cells having the modification of reduced or inhibited expression of SIT1, BST2, and PD-1 may be of any kind of immune cells, but in specific embodiments they are lymphoid cells including T cells of any kind, tumor-infiltrating lymphocytes, or a mixture thereof.

Embodiments of the disclosure provide for one or more adoptive cell therapy compositions for treatment of any cancer. In specific embodiments, the cellular compositions comprise modified TILs and include formulations for administration to an individual in need of cancer treatment. The compositions may or may not be formulated for storage, transport, and/or delivery.

Embodiments of the disclosure include cells for immunotherapy that include T cells or TILs that are engineered to be more effective at cancer treatment compared to T cells or TILs that lack the same or similar modification(s). In some embodiments, the TILs are engineered to have reduction or elimination of expression of SIT1, BST2, and PD-1. Such engineering may occur by any suitable means. Thus, the TILs may be gene edited, and the gene editing may occur by any means. The gene editing may or may not be transient; in specific cases the gene editing is permanent.

In some embodiments, the cells are derived from the blood, bone marrow, lymph, umbilical cord, or lymphoid organs. In some aspects, the cells are human cells. In some cases the cells are primary cells, such as those isolated directly from a subject and/or isolated from a subject and frozen. The cells may or may not be expanded. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the methods include isolating cells from a subject, preparing, processing, culturing, engineering them to have reduced or eliminated expression of SIT1, BST2, and PD-1, optionally engineering them to express one or more engineered antigen receptors, (and the order of the aforementioned actions may not be as listed) and re-introducing them into the same and/or different subject, optionally before or after cryopreservation.

Among the sub-types and subpopulations of T cells (e.g., CD4+ and/or CD8+ T cells) are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the engineered T cell populations is enriched for or depleted of cells that are positive for one or more specific markers, such as surface markers. In some embodiments, one or more of the engineered T cell populations is enriched for or depleted of cells that are negative for one or more specific markers, such as surface markers. In some cases, such markers are those that are absent or expressed at relatively low levels on certain populations of T cells (e.g., non-memory cells) but are present or expressed at relatively higher levels on certain other populations of T cells (e.g., memory cells). In some cases, any cell population may comprise of mix of cells having enrichment of cells positive for one or more specific markers and having enrichment of cells negative for one or more specific markers.

In some embodiments, T cells are separated from a PBMC sample by negative selection of markers expressed on non-T cells, such as B cells, monocytes, or other white blood cells. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In specific embodiments, the immune cells are CD8+ cells, including CD8+ T cells.

In some embodiments, CD8+ T cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such subpopulations.

In some embodiments, the engineered T cells are autologous T cells with respect to a recipient individual. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests may be cultured with one or more specific interleukins, such as IL-2. The T cells may then be engineered to have disruption of expression of SIT1, BST2, and PD-1 and, optionally engineered in one or more other modifications, such as expression of one or more engineered antigen receptors. The cells produced from such methods may be provided back to the individual from which the cancer cells were originally obtained and/or may be provided to another individual.

Any cells, including any cultured cells, can be expanded, and in some cases cells can be pooled and expanded. Expansion provides an increase in the number of desired cells of at least about 50-fold (e.g., 50-, 60-, 70-, 80-, 90-, or 100-fold, or greater) over a period of about several hours to about 21 days or any range therebetween, days in some cases. For example, the range may be any one of 1, 2, 3, 4, 5, 6, 7, 8, 9, 10, 11, 12, 13, 14, 15, 16, 17, 18, 19, 20, 21, 22, or 23 hours to any one of 1, 2, 3, 4, 5, 6, 7, 8, 9, 10, 11, 12, 13, 14, 15, 16, 17, 18, 19, 20, or 21 days and any range therebetween. In particular cases, expansion provides an increase of at least about 100-fold (e.g., 100-, 200-, 300-, 400-, 500-, 600-, 700-, 800-, 900-, or greater) over a period of about 10 to about 14 days. TIL may be expanded about 1-21 days with any range of time for stimulation (either antibody plate-bound±one or more cytokines) before or after genetic modification. Antibody stimulation (whether beads- or plate-bound) and T cells may be cultured together for any point in time between 1-21 days, and then stimulation may be removed and continuation (or not) of cell expansion would occur in appropriate medium with or without one or more cytokines. Recurrent stimulation may be performed such that T cells are cultured for 60 days or more.

### III. Methods of Preparing the Cells

Embodiments of the disclosure include methods wherein one or more types of lymphoid cells are obtained and engineered to have disruption of expression of SIT1, BST2, and PD-1. In particular embodiments, the cells are expanded prior to and/or after the disruption of expression. The cells to be engineered may be T cells, TILs, or a mixture thereof, in some cases. In certain embodiments, the cells are prepared by also engineering them to express one or more other heterologous nucleic acids, such as one or more engineered antigen receptors. The order of disruption of (1) SIT1, (2) BST2, and (3) PD-1 may be of any order and by any method. In some cases, the order is (1), (2), (3); (1), (3), (2); (2), (1), (3); (2), (3), (1); (3), (1), (2); or (3), (2), (1). In specific embodiments, disruption of expression of SIT1, BST2, and PD-1 occurs prior to modifying the cells to expression one or more other heterologous nucleic acids, whereas in other embodiments disruption of expression of SIT1, BST2, and PD-1 occurs subsequent to modifying the cells to expression one or more other heterologous nucleic acids. In some cases, cells previously modified to express one or more other heterologous nucleic acids (including CARs and/or T cell receptors and/or cytokines) are obtained elsewhere, such as from a repository, and prepared to have disruption of expression of SIT1, BST2, and PD-1. In some cases, cells previously modified to have disruption of expression of SIT1, BST2, and PD-1 are obtained elsewhere, such as from a repository, and prepared to express one or more other heterologous nucleic acids (including CARs and/or T cell receptors and/or cytokines). The cytokine(s) may be heterologous and may be integrated; specific examples include at least IL-2, IL-7, IL-21, and/or IL-15.

A. Gene Editing

In particular embodiments, cells for adoptive cell therapy are produced upon modification of the cells to have disrupted expression, including reduced or inhibited expression, of SIT1, BST2, and PD-1, thereby improving the cells for the adoptive cell therapy. The modification of the cells may occur by any particular suitable method, but in specific embodiments the modification occurs by CRISPR.

In some embodiments, the gene disruption is carried out by effecting a disruption in one or both of the desired genes, such as a knock-out, insertion, missense or frameshift mutation, including biallelic frameshift mutation, deletion of all or part of the gene, e.g., one or more exon or portion therefore, and/or knock-in, as some examples. In certain cases, the disruption can be affected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the SIT1, BST2, and PD-1 genes or a portion thereof.

In some embodiments, SIT1, BST2, and/or PD-1 gene disruption is performed by induction of one or more double-stranded breaks and/or one or more single-stranded breaks in the gene, including in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

In some embodiments, gene disruption is achieved using antisense techniques, including by RNA interference (RNAi), short interfering RNA (siRNA), short hairpin (shRNA), and/or ribozymes are used to selectively suppress or repress expression of the gene. siRNA technology is RNAi that employs a double-stranded RNA molecule having a sequence homologous with the nucleotide sequence of mRNA that is transcribed from the gene, and a sequence complementary with the nucleotide sequence. siRNA generally is homologous/complementary with one region of mRNA that is transcribed from the gene, or may be siRNA including a plurality of RNA molecules that are homologous/complementary with different regions. In some aspects, the siRNA is comprised in a polycistronic construct.

In some embodiments, the disruption is achieved using a DNA-targeting molecule, such as a DNA-binding protein or DNA-binding nucleic acid, or complex, compound, or composition, containing the same, which specifically binds to or hybridizes to the SIT1 gene or the PD-1 gene or the BST2 gene, respectively. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data.

In cases where gene alteration is carried out by induction of one or more double-stranded breaks and/or one or more single-stranded breaks in the gene, the double-stranded or single-stranded breaks may undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The T cells or TILs may be introduced to a guide RNA and CRISPR enzyme, or mRNA encoding the CRISPR enzyme. For CRISPR-mediated disruption, the guide RNA and endonuclease may be introduced to the T cells or TILs by any means known in the art to allow delivery inside cells or subcellular compartments of agents/chemicals and molecules (proteins and nucleic acids) can be used including liposomal delivery means, polymeric carriers, chemical carriers, lipoplexes, polyplexes, dendrimers, nanoparticles, emulsion, natural endocytosis or phagocytose pathway as non-limiting examples, as well as physical methods such as electroporation. In specific aspects, electroporation is used to introduce the guide RNA and endonuclease, or nucleic acid encoding the endonuclease.

In one exemplary method, the method for CRISPR knockout of multiple genes may comprise isolation of T cells or TILs from a cancer of the individual, including from a tumor. When obtained from an individual for autologous purposes, the T cells or TILs may be obtained by any suitable method such as through biopsy or routine sample collection of any kind, including from blood, bone marrow, and so forth. In cases wherein the T cells or TILs are allogeneic with respect to a recipient individual, the source of the T cells or TILs may be from storage, from a commercial source, fresh from a donor, and so forth.

In embodiments where the T cells or TILs are expanded, they may be expanded by any suitable method, such as initial expansion of T cells or TILs from tumor fragments via culture in IL-2 followed by a rapid expansion protocol involving stimulation via CD3 crosslinking and IL-2 with or without additional co-stimulation through 4-1BB/CD137 in the presence of peripheral blood mononuclear cells (PBMCs) or artificial antigen presenting cells. In specific embodiments, any expansion protocol may be with or without additional co-stimulation through but not limited to 4-1BB/CD137, for example. Particular embodiments include an expansion protocol as described in Tavera et al., J. Immunother. 2018; 41(9):399-405, which is incorporated by reference herein in its entirety. This improved TIL expansion protocol combines the required three signals for T-cell activation in addition to co-culturing with tumor fragments. The protocol uses OKT3 antibody (Anti-CD3) for TCR stimulation as signal 1, CD127/4-1BB agonistic antibody (anti-4-1BB, Urelumab) as signal 2, and high dose IL-2 as signal 3.

Prior to or following expansion, the T cells or TILs may be subject to engineering to effect knockdown or knockout of SIT1 and/or BST2 and/or PD-1. In cases wherein CRISPR is utilized, the engineering of SIT1 and/or BST2 and/or PD-1 may occur in the same electroporation step or in successive electroporation steps. When the electroporation steps are successive, the knockout/knockdown of one or more of SIT1, BST2, and PD-1 may be before or after the knockout/knockdown of BST2, PD-1, and SIT1, respectively. Any combination of knockout/knockdown of BST2, PD-1, and SIT1 may occur in any order, for example where each of the three genes are edited in a combination two or three electroporation steps. As one specific example only, BST1 and PD-1 may be edited in a first electroporation step, and SIT1 may be edited in a second electroporation step (and any combination thereof). Following CRISPR editing of the T cells or TILs, they may or may not be subjected to an additional expansion step, for example through re-stimulation via CD3 crosslinking and IL2 stimulation. Any expansion step may be with or without re-stimulation via CD3 crosslinking. Any expansion step could be with or without co-stimulation via 4-1BB or others. Any expansion step could be with or without cytokine stimulation (such as with IL-2, IL-7, IL-15, a combination thereof, or others).

In some aspects, a Cas nuclease and gRNA (including a fusion of crRNA specific for the target sequence and fixed tracrRNA) are introduced into the T cells or TIL. Any CRISPR methods may utilize either single guide-RNA (sgRNA) or a duplex of tracrRNA and crRNA. In general, target sites at the 5′ end of the gRNA target the Cas nuclease to the target site, e.g., the BST2 gene or the PD-1 gene or the SIT1 gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

In some embodiments, the alteration of the expression, activity, and/or function of the BST2 and/or the SIT1 gene and/or the PD-1 gene is carried out by disrupting the corresponding gene. In some aspects, the gene is modified so that its expression is reduced by at least at or about 20, 30, or 40%, generally at least at or about 50, 55, 60, 65, 70, 75, 80, 85, 90, 91, 92, 93, 94, 95, 96, 97, 98, 99, or 100% as compared to the expression in the absence of the gene modification or in the absence of the components introduced to effect the modification.

B. Procurement and Expansion

The cells that are manipulated to have disrupted expression of SIT1, BST2, and PD-1 may be autologous or allogeneic with respect to a recipient individual. Regardless if the cells are autologous or allogeneic, the cells to be manipulated may or may not come from a repository. In some cases, the cells are obtained from an individual to be treated, manipulated as described herein, and delivered back into the same individual. In some cases, cells are manipulated as described herein and stored in a repository before being delivered to an individual in need thereof; in such cases, the cells may or may not be further manipulated.

In cases wherein cancer cells are obtained from an individual and manipulated as described herein, the cells may be obtained as a biopsy, for example, using any standard technique in the art, such as punch biopsy, needle biopsy, CT-guided biopsy, Ultrasound-guided biopsy, aspiration biopsy, and so forth. In some cases, a sample from a biopsy is analyzed for cancer diagnosis and/or prognosis (including determining the type and/or stage of the cancer and/or particular marker(s) of the cancer), and cells from the same biopsy are manipulated as described herein, such as following the cancer diagnosis and/or prognosis. In other cases, cells from a different biopsy as a biopsy that facilitated cancer diagnosis and/or prognosis are used to generate the adoptive cell therapy.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example. One specific example of an expansion protocol is described in Tavera et al. (J. Immunotherap., vol. 41, 9 (2018), which is incorporated by reference herein in its entirety.

C. Engineered Antigen Receptors

In certain embodiments, the cells having disrupted expression of BST2, SIT1, and PD-1 are also modified to express one or more engineered antigen receptors. In particular embodiments, the T cells or TILs are manipulated to express one or more heterologous engineered antigen receptors, such as engineered TCRs, CARs, chimeric cytokine receptors, chemokine receptors, a combination thereof, and so on. The engineered antigen receptors are synthetically generated by the hand of man. In particular embodiments, the T cells or TILs are modified to express a CAR and/or TCR having antigenic specificity for one or more cancer antigens. Multiple CARs and/or TCRs, such as to different antigens, may be added to the T cells. In some aspects, the T cells or TILs are engineered to express the CAR or TCR by knock-in of the CAR or TCR at a particular gene locus, such as by using CRISPR, for example.

1. T Cell Receptors

In some embodiments, the engineered heterologous antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3 chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

2. Chimeric Antigen Receptors (CARs)

In some embodiments, the T cells are engineered to express one or more CARs comprising an extracellular antigen-recognition domain that specifically binds to one or more antigens. In some embodiments, the antigen is a protein expressed on the surface of cells.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

In some embodiments, the CAR comprises: a) one or more intracellular signaling domains, b) a transmembrane domain, and c) one or more extracellular domains (the CAR may be bi-specific or tri-specific, in some cases) comprising one or more antigen binding regions.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the disclosure includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3zeta, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. In certain embodiments, the CAR may be co-expressed with a cytokine to improve persistence when there is a low amount of tumor-associated antigen. For example, CAR may be co-expressed with one or more cytokines, such as IL-7, IL-2, IL-15, IL-12, IL-18, IL-21, or a combination thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

3. Antigens

Among the antigens targeted by the genetically engineered heterologous antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas, and cancers having solid tumors. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP). Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STATS, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, BST2, and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### IV. Methods of Use of the Cells

Embodiments of the disclosure include improved cellular immunotherapy methods of treating or preventing any kind of cancer, including hematological malignancies or solid tumors, by using the engineered immune cells (TILs and/or T cells, as examples) as at least part of the therapy. In particular embodiments, an effective amount of the engineered immune cells for the adoptive cell therapy are provided to an individual in need thereof. The TILs and/or engineered T cells may be autologous with respect to the individual, although in some cases the TILs and/or engineered T cells are allogeneic with respect to the individual.

The disclosure provides approaches to improvements on cancer immunotherapy, particularly with respect to use of engineered TILs and/or engineered T cells. In particular embodiments, knocking out or knocking down BST2, PD-1, and SIT1 in TILs and/or T cells overcomes immunosuppression in the tumor microenvironment. In specific embodiments, the knocking out or down of BST2, PD-1, and SIT1 facilitates immunosuppression in the tumor microenvironment because it allows the engineered TILs and/or T cells to circumvent one or more inhibitory signals in or from the tumor microenvironment.

In particular aspects of the disclosure, TILs and/or T cells are used for an individual that has one or more tumors. In certain embodiments, TILs and/or engineered T cells are obtained from the individual in need of cancer therapy, such as obtained from an individual's own cancer (including in the form of a tumor). The individual may be known to have cancer and have cells taken from the cancer for obtaining T cells or TILs. In other cases, an individual may not be known to have cancer, and the T cells or TILs are obtained from tissue being analyzed for the cancer (for example, by biopsy). In other cases, T cells or TILs are obtained when the individual does not have cancer and, in some cases, is deposited in a repository (and in which case, the engineering may happen before and/or after placement in the repository). In any case, T cells or TILs may be taken from the cancer of an individual, expanded to a suitable number of expanded respective T cells or TILs, engineered to have knockout or knockdown of SIT1 and/or BST2 and/or PD-1, and delivered back into the individual from which the TILs were originally obtained (or alternatively given to another individual, or both).

The cancer being treated in the individual may be of any kind, including hematological malignancies or cancers with solid tumors. Hematological malignancies include at least cancers of the bone marrow, T or B cell malignancies, leukemias, lymphomas, blastomas, myelomas, and the like. Specific examples include at least Acute myeloid leukemia, B-cell acute lymphoblastic leukemia, T-cell acute lymphoblastic leukemia, Myelodysplastic syndromes, Chronic lymphocytic leukemia/small lymphocytic lymphoma, Follicular lymphoma, Lymphoplasmacytic lymphoma, Diffuse large B-cell lymphoma, Mantle cell lymphoma, Hairy cell leukemia, Plasma cell myeloma or multiple myeloma, Mature T/NK neoplasms, and so forth. Examples of solid tumors include tumors of the brain, lung, breast, prostate, pancreas, stomach, anus, head and neck, bone, skin, liver, kidney, thyroid, testes, ovary, endometrium, gall bladder, peritoneum, cervix, colon, rectum, vulva, spleen, a combination thereof, and so forth.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Methods of the disclosure encompass immunotherapies including adoptive cellular therapy, with TILs (whether expanded or not) and/or T cells (whether expanded or not) for treating cancer, where the immunotherapies are improved to allow greater efficacy for the immunotherapy by inhibiting expression of BST2, SIT1, and PD-1.

Provided herein are methods for treating or delaying progression of cancer in an individual comprising administering to the individual an effective amount of adoptive cell therapy that may in particular cases be antigen-specific. In such cases, the treatment for the individual is both targeted to one or more desired antigens and is more effective at a tumor site because it has disrupted expression of BST2, PD-1, and SIT1.

In particular embodiments of the present disclosure, an effective amount of TILs and/or T cells are delivered to an individual in need thereof, such as an individual that has cancer of any kind. The cells then enhance the individual's immune system to attack the cancer cells. In some cases, the individual is provided with one or more doses of the TILs and/or T cells. In cases where the individual is provided with two or more doses of the TILs and/or T cells, the duration between the administrations should be sufficient to allow time for propagation in the individual, and in specific embodiments the duration between doses may be 1, 2, 3, 4, 5, 6, 7, or more days. Successive doses may or may not be identical in amount to one another. In some cases, the successive doses decrease over time or increase over time.

Specific methods of the disclosure encompass delivery of an effective amount of compositions comprising TILs and/or T cells engineered for knockout or knockdown of BST2, PD-1, and SIT1. In some cases, there is a population of multiple cells, each of which single TILs and/or single T cells have knockout of BST2, PD-1, and SIT1. In cases wherein an order of delivery of the two or more components is desired, the order may be of any kind so long as the delivery is therapeutically effective. In specific embodiments, delivery of TIL cells and/or T cells comprising knockout or knockdown of BST2, PD-1, and SIT1 occurs prior to a second therapy so that the second therapy becomes more effective than without the initial TIL and/or T cell step(s). In some cases, delivery of TIL cells and/or T cells comprising knockout or knockdown of BST2, PD-1, and SIT1 occurs before and/or after any one or more additional cancer therapies, including surgery, chemotherapy, drug therapy, radiation, immunotherapy, hormone therapy, or a combination thereof.

### V. Pharmaceutical Compositions

Pharmaceutical compositions of the present disclosure comprise an effective amount of engineered cells (including engineered TILs and/or engineered T cells, merely as examples) having modified expression of BST2, PD-1, and SIT1 (and/or reagents to generate same ex vivo or in vivo) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises engineered TILs and/or engineered T cells (and/or reagents to generate same ex vivo or in vivo) will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intracranially, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The engineered TILs and/or engineered T cells (and/or reagents to generate same ex vivo or in vivo) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include the engineered TILs and/or engineered T cells (and/or reagents to generate same ex vivo or in vivo), and optionally an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the engineered TILs and/or engineered T cells (and/or reagents to generate same ex vivo or in vivo) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

An amount of cells for a therapeutically effective purpose may be any amount determined by a medical provider taking into consideration the individual's age, weight, physical fitness, tumor size or load, stage of the disease (e.g., metastasis), and/or any additional health condition. The dose may also be determined based on any additional regimen an individual is undergoing (e.g, combination therapy) since safety profiles and tolerance of the individual drugs (and other criteria) may affect the decision. In specific embodiments, a therapeutically effective amount of cells is any dosage between 1×10⁴ to 5×10⁹ cells/kg body weight and any range therebetween. Multiple cycles of any dose therein may be administered to the individual. One example of an administration technique is as described in Rosenberg et al. (1988).

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In particular embodiments of the present disclosure, the engineered TILs and/or engineered T cells (and/or reagents to generate same ex vivo or in vivo) are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In certain embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other particular embodiments of the disclosure, the active compound engineered TILs or engineered T cells (and/or reagents to generate same ex vivo or in vivo) may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VI. Combination Therapies

In certain embodiments, the compositions and methods of the present embodiments involve a cancer therapy that is additional to the compositions comprising the exemplary engineered TILs and/or engineered T cells. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent(s). The additional therapy may be one or more of the chemotherapeutic agents known in the art.

An immune cell therapy (in addition to the TIL therapy and/or engineered T cell therapy of the disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from the composition(s) of the disclosure, one would generally ensure that a significant period of time did not expire between the time of each delivery, such that the two compounds would still be able to exert an advantageously combined effect on the patient. In such instances, it is contemplated that one may provide a patient with the immunotherapy therapy and the disclosed compositions within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies (outside of the disclosed TIL cell therapy and/or engineered T cell therapy) may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells other than those having knockdown or knockout of TGFBR2 and/or TIGIT.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons of any kind, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. Kits of the Disclosure

Any of the compositions described herein may be comprised in a kit. In a non-limiting example, (and/or reagents to generate same), and these may be comprised in suitable container means in a kit of the present disclosure.

The compositions of the kits may be packaged either in aqueous media or in lyophilized form. The container means of the kits will generally include at least one vial, test tube, flask, bottle, syringe or other container means, into which one or more components may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the engineered TILs and/or engineered T cells (and/or reagents to generate same), and any other reagent containers, in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly envisioned. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle. In some embodiments, reagents or apparatuses or containers are included in the kit for ex vivo use.

### EXAMPLES

The following examples are included to demonstrate particular embodiments of the invention. It should be appreciated by those of skill in the art that the techniques disclosed in the examples that follow represent techniques discovered by the inventors to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosed subject matter.

### Example 1

### Modulation of Novel T Cell Surface Proteins to Enhance Adoptive T Cell Therapy

To identify genes whose products whose expression may be useful to modify to enhance adoptive T cell therapy, a bioinformatics screen was performed that identified clinically relevant CD8 T cell surface molecules. Specifically, in order to find relevant surface molecules on cytotoxic CD8+ T cells, the inventors implemented a bioinformatics approach that identified 47 molecules from 31 tumor biopsies of metastatic melanoma patients that were non-responders to anti-PD-1 and/or anti-CTLA-4. Within the 47 hits, the inventors found SIT1, BST2, and PD1 to be overexpressed in CD8+ T cells from non-responders. This data was obtained from multiple data sets that allow prioritization of top genes from non-responders group from Sade-Feldman et al., 2018.

SIT1, a novel surface protein only present in some immune cells such as T cells and B cells has been reported to regulate negatively T-cell antigen receptor (TCR)-mediated signaling, but the exact mechanism is not well described. However, SIT1-deficient mice T cells are hypersensitive because of reduced inhibitory signals. On the other hand, BST2 has been well-studied in the virology field, as well as in the context of being a tumor target because of its overexpression in many cancers. The role of BST2 in CD8+ T cells within the tumor microenvironment is not well-described. In embodiments of the disclosure, these two molecules, unlike PD-1, are useful as modification targets for immunotherapy. As demonstrated herein, their downregulation in T cells increases their tumor killing and even more so in combination with PD-1.

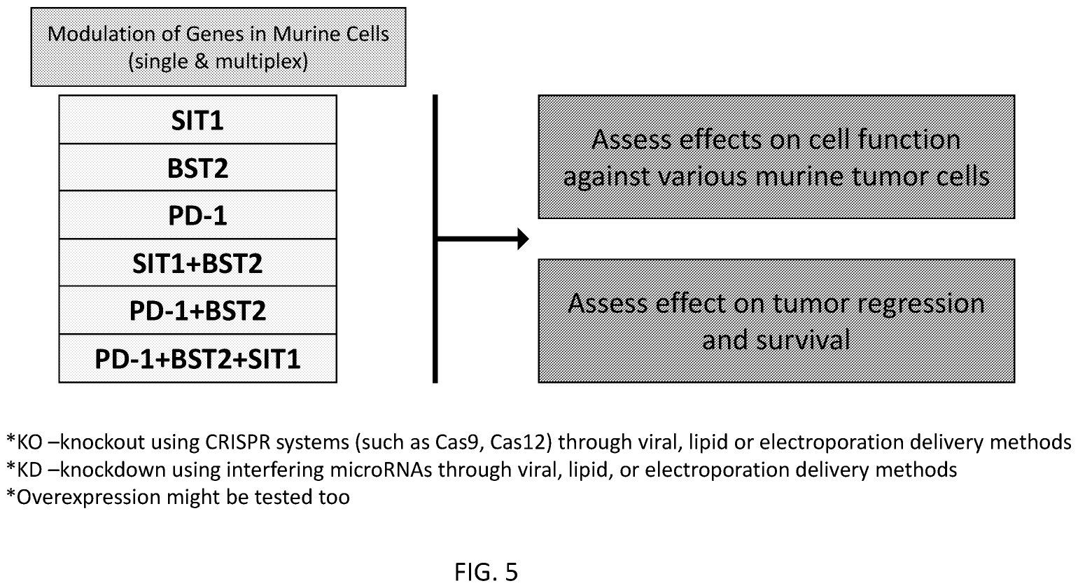

In one embodiment, murine validation studies may be performed in vitro and in vivo, including for CD8+ T cells. As illustrated for one approach in FIG. 5, one or multiple of SIT1, BST2, and PD-1 are modulated, and cytotoxicity levels are assessed, such as against paired tumor cells. In specific cases, these genes may be modulated by knockout, such as using CRISPR systems (such as Cas9, Cas12) through viral, lipid or electroporation delivery methods. In some cases, the gene or genes may be knocked down, such as using interfering microRNAs through viral, lipid, or electroporation delivery methods. In certain cases, overexpression of one or combinations of the genes may alternatively or additionally be studied.

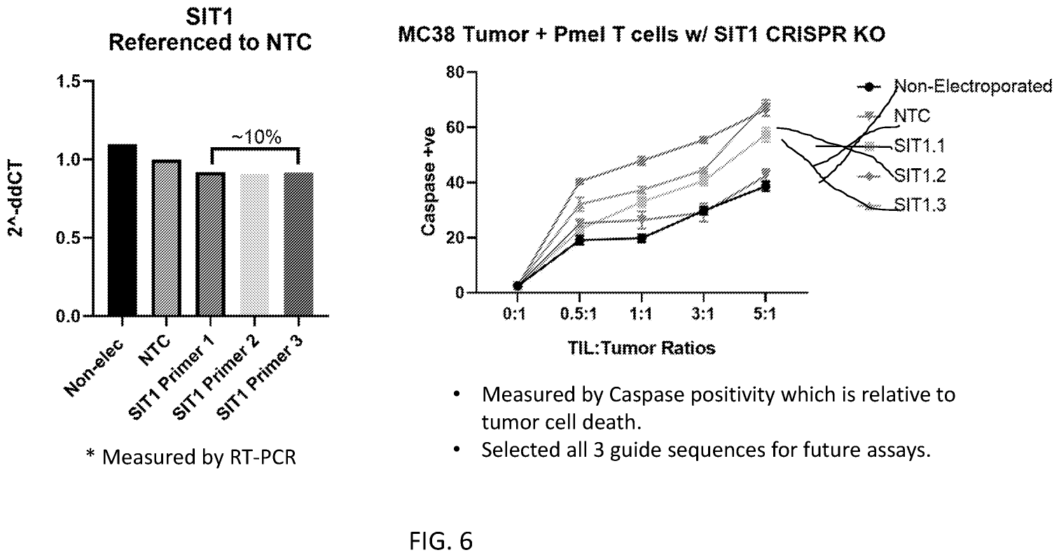

FIG. 6 shows that in vitro CRISPR/Cas9 knockout of SIT1 resulted in C57BL/6 mouse CD8+ T cells resulted in significant killing, compared to control, against MC38 mouse colon tumor line. The mouse T cells recognize the gp100/pmel antigen (melanoma antigen) that is expressed on MC38 tumor cells. MC38 tumor cells were modified to express this antigen in order to be recognized by the T cells.

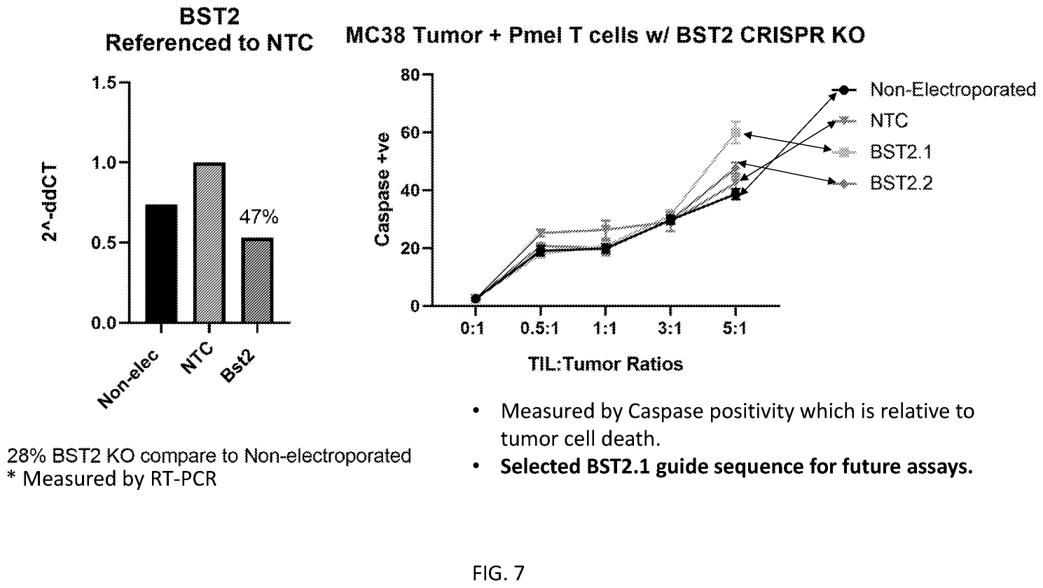

FIG. 7 shows that in vitro CRISPR/Cas9 knockout of BST2 resulted in significant killing compared to controls using analogous methods as in FIG. 6.

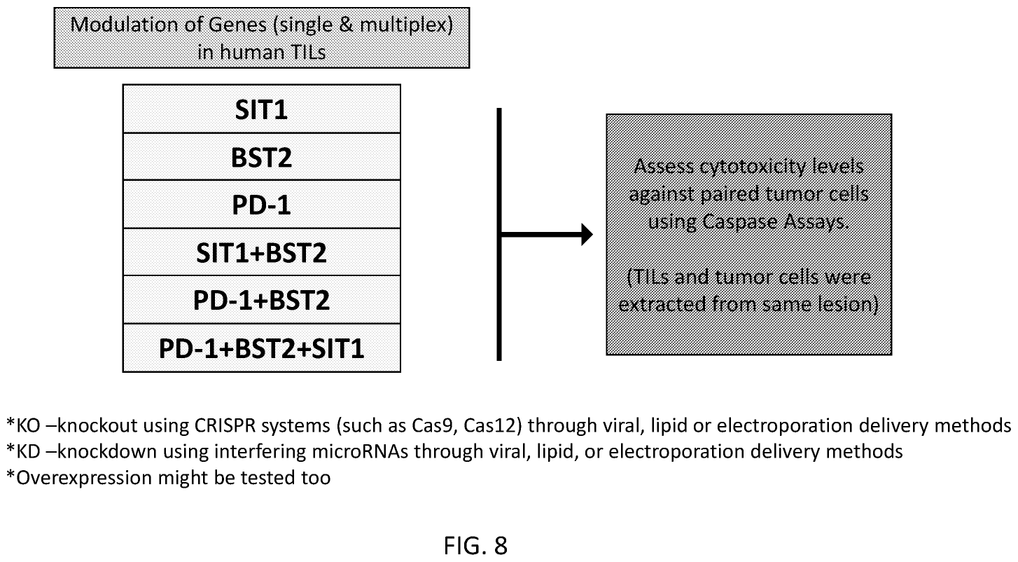

In certain embodiments, validation studies are performed for human TILs. As noted in FIG. 8, one or multiple of SIT1, BST2, and PD-1 are modulated, and cytotoxicity levels are assessed, such as against paired tumor cells. In specific cases, these genes may be modulated by knockout, such as using CRISPR systems (such as Cas9, Cas12) through viral, lipid or electroporation delivery methods. In some cases, the gene or genes may be knocked down, such as using interfering microRNAs through viral, lipid, or electroporation delivery methods. In certain cases, overexpression of one or combinations of the genes may alternatively or additionally be studied.

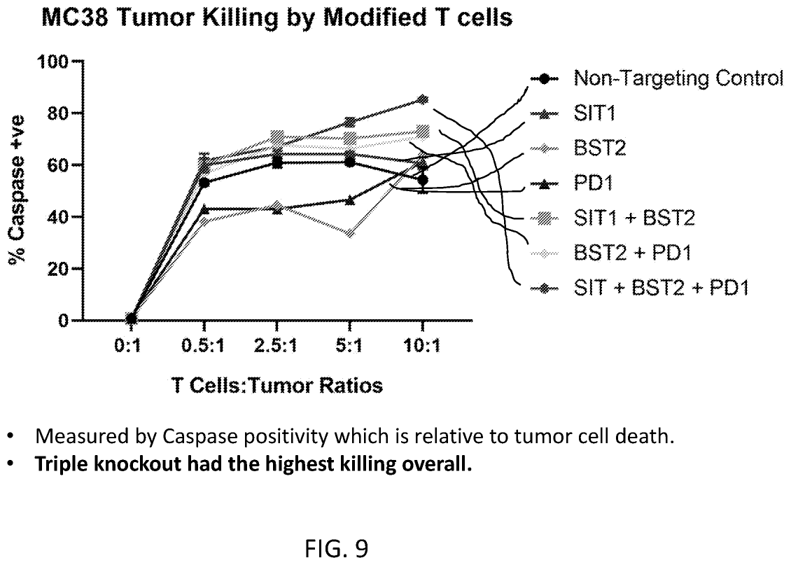

As illustrated in FIG. 9, in vitro multiplex CRISPR/Cas9 knockout of SIT1, BST2, and PD-1 in mouse CD8+ T cells resulted in significant killing, compared to control and single knockout, against MC38 mouse colon tumor line.

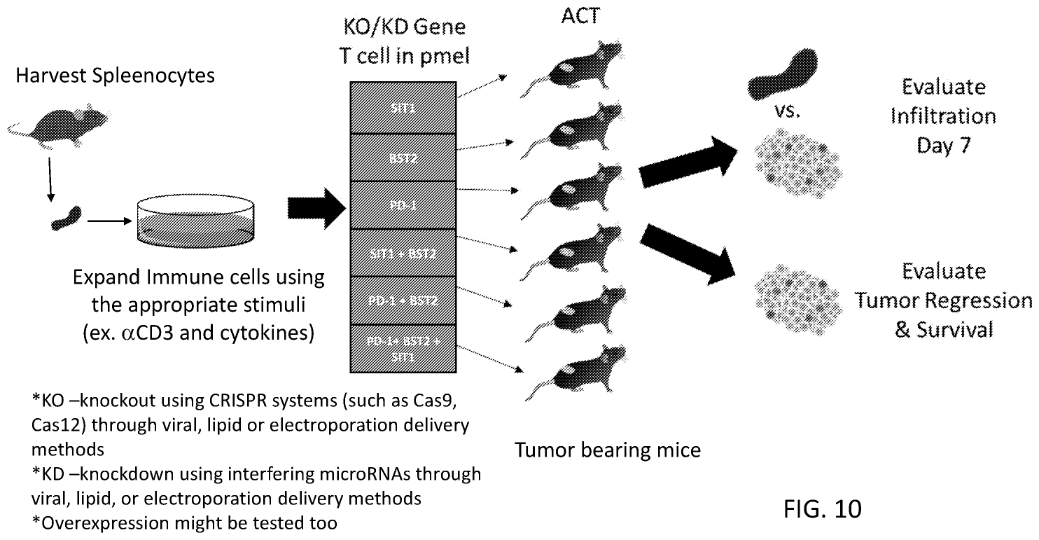

In some cases, murine validation studies are performed in vivo. FIG. 10 provides one such example wherein B 16 melanoma cells are injected into mice to generate mice with tumors. T cells harvested from mouse spleens are activated and subjected to knockout or knockdown of one of the genes or combinations of the genes. The modified T cells are provided to tumor bearing mice through intravenous tail injection. Following a suitable duration of time, tumor regression and survival of the recipient mice are evaluated. Infiltration of lymphoid cells in the tumors may additionally or alternatively be evaluated.

### REFERENCES

Arndt, B. et al. (2011). PLOS One 6(9): 323761.
Austin-Ward and Villaseca, 1998
Bego, et al., (2019). J. Biol. Chem. 294(27):10503-10518.
Bukowski et al., Clinical Cancer Res., 4(10):2337-2347, 1998.
Chothia et al., EMBO J. 7:3745, 1988.
Cohen et al. J Immunol. 175:5799-5808, 2005.
Davidson et al., J. Immunother., 21(5):389-398, 1998.
Davila et al. PLoS ONE 8(4): e61338, 2013.
Guo, X. et al. (2018). Nat Med 24: 978-985.
Hanibuchi et al., Int. J. Cancer, 78(4):480-485, 1998.
Hellstrand et al., Acta Oncologica, 37(4):347-353, 1998.
Hollander, Front. Immun., 3:3, 2012.
Hui and Hashimoto, Infection Immun., 66(11):5329-5336, 1998.
Janeway et al, Immunobiology: The Immune System in Health and Disease, 3^{rd} Ed., Current Biology Publications, p. 433, 1997.
Jores et al., PNAS U.S.A. 87:9138, 1990.
Kabat et al., “Sequences of Proteins of Immunological Interest, US Dept. Health and Human Services, Public Health Service National Institutes of Health, 1991, 5^{th} ed.
Leal, M., Ann N Y Acad Sci 1321, 41-54, 2014.
Lefranc et al., Dev. Comp. Immunol. 27:55, 2003.
Li et al. Nat Biotechnol. 23:349-354, 2005.
Marie-Cardine, A., et al. (1999). J. Exper Med. 189(8), 1181-1194.
Parkhurst et al. Clin Cancer Res. 15: 169-180, 2009.
Pfrepper, K.-I. et al. (2001). Eur. J. Immunol. 31: 1825-1836.
Qin et al., Proc. Natl. Acad. Sci. USA, 95(24):14411-14416, 1998.
Remington: The Science and Practice of Pharmacy, 21^{st} Ed. Lippincott Williams and Wilkins, 2005,
Remington's Pharmaceutical Sciences, 18th Ed. Mack Printing Company, 1990, pp. 1289-1329
Rosenberg, et al. (1988). New Engl J. Med. 319:1676.
Sade-Feldman et al., 2018, Cell 175, 998-1013
Sadelain et al., Cancer Discov. 3(4): 388-398, 2013.
Singh et al., Cancer Research, 68:2961-2971, 2008.
Singh et al., Cancer Research, 71:3516-3527, 2011.
Tavera, et al. (2018). J. Immunother. 41(9):399-405.
Turtle et al., Curr. Opin. Immunol., 24(5): 633-39, 2012.
Varela-Rohena et al. Nat Med. 14: 1390-1395, 2008.
Wu et al., Cancer, 18(2): 160-75, 2012.
Yi, E. et al. (2019). Biochem. Biophys. Res. Commun. 509(2), 414-420.
U.S. Pat. No. 4,870,287
U.S. Pat. No. 5,580,579
U.S. Pat. No. 5,641,515
U.S. Pat. No. 5,739,169
U.S. Pat. No. 5,760,395
U.S. Pat. No. 5,792,451
U.S. Pat. No. 5,801,005
U.S. Pat. No. 5,824,311
U.S. Pat. No. 5,830,880
U.S. Pat. No. 5,846,945
U.S. Pat. No. 6,410,319
U.S. Pat. No. 6,451,995
U.S. Pat. No. 6,544,518
U.S. Pat. No. 7,070,995
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 8,252,592
U.S. Pat. No. 8,324,353
U.S. Pat. No. 8,339,645
U.S. Pat. No. 8,398,282
U.S. Pat. No. 8,479,118
U.S. Patent Application Publication US2002/131960,
U.S. Patent Application Publication US2013/0149337
U.S. Patent Application Publication US2013/0287748
WO2000/14257
WO2012/129514
WO2013/071154
WO2013/123061
WO2013/126726
WO2013/166321
WO2014/031687
WO2014/055668
European patent application number EP2537416

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A composition comprising at least one engineered immune cell, wherein the cell is engineered to have disruption in expression of the following endogenous genes:
(a) Signaling Threshold Regulating Transmembrane Adaptor 1 (SIT1),
(b) Bone Marrow Stromal Cell Antigen 2 (BST2), and
(c) Programmed cell death protein 1 (PD-1).

2. The composition of claim 1, wherein the immune cell is a lymphoid cell.

3. The composition of claim 2, wherein the lymphoid cell is a tumor-infiltrating lymphocyte (TIL), T cell, B cell, or a mixture thereof.

4. The composition of claim 3, wherein the immune cell is a TIL or T cell.

5. The composition of any one of claims 1-4, wherein the cell is obtained from an individual in need of cell therapy.

6. The composition of claim 5, wherein the individual has cancer.

7. The composition of claim 6, wherein the cell is obtained from the cancer of the individual.

8. The composition of any one of claims 1-4, wherein the cells are obtained from a repository.

9. The composition of any one of claims 1-8, wherein the composition comprises a population of the engineered immune cells.

10. The composition of any one of claims 1-9, wherein the cells are autologous with respect to a recipient individual.

11. The composition of any one claims 1-9, wherein the cells are allogeneic with respect to a recipient individual.

12. The composition of any one claims 1-11, wherein the disruption in expression comprises a knockout of SIT1, BST2, and PD-1.

13. The composition of any one of claims 1-11, wherein the disruption in expression comprises a knockdown of SIT1, BST2, and PD-1.

14. The composition of any one of claims 1-13, wherein the disruption in expression is a knockout of one or two of SIT1, BST2, and PD-1, and the disruption in expression is a knockdown of the respective remaining two or one of SIT1, BST2, and PD-1.

15. The composition of any one of the preceding claims, wherein a disruption in expression is the result of CRISPR gene editing.

16. The composition of any one of the preceding claims, wherein the immune cell is a TIL or T cell that is positive for cluster of differentiation 8 (CD8).

17. The composition of any one of the preceding claims, wherein when the cells are T cells or TILs, they have disruption in expression of the native T cell receptor.

18. The composition of any one of the preceding claims, wherein the engineered cell further comprises a disruption in expression in one or more inhibitory molecules.

19. The composition of claim 18, wherein the inhibitory molecule is cytotoxic T-lymphocyte-associated protein 4 (CTLA4), T-cell immunoglobulin mucin-3 (TIM-3), Lymphocyte Activating 3 (LAG3), or a combination thereof.

20. The composition of any one of the preceding claims, wherein the cells further comprise expression of one or more engineered receptors.

21. The composition of claim 20, wherein the engineered receptor is an engineered antigen receptor.

22. The composition of claim 21, wherein the engineered antigen receptor is a chimeric antigen receptor or an engineered T cell receptor.

23. The composition of any one of the preceding claims, further comprising a pharmaceutically acceptable carrier.

24. A method of producing the cell or cells of any one of claims 1-23, comprising the step of subjecting one or more immune cells to disruption of expression of the endogenous SIT1, BST2, and PD-1 genes in the immune cell or cells.

25. The method of claim 24, wherein the disruption is performed by CRISPR gene editing.

26. The method of claim 24 or 25, wherein prior to the subjecting step, the cells are expanded.

27. The method of any one of claims 24-26, wherein the immune cell or cells is obtained from blood.

28. The method of claim 27, wherein the blood is peripheral blood.

29. The method of any one of claims 24-28, wherein the immune cell is obtained from a tumor.

30. The method of any one of claims 24-29, further comprising the step of expanding the cells.

31. The method of claim 30, wherein the expanding occurs prior to the disruption of expression.

32. The method of claim 30, wherein the expanding occurs subsequent to the disruption of expression.

33. The method of any one of claims 24-32, further comprising modifying the cells to express one or more engineered receptors.

34. The method of claim 33, wherein modifying the cells to express one or more engineered receptors occurs prior to the disruption of expression.

35. The method of claim 33, wherein modifying the cells to express one or more engineered receptors occurs subsequent to the disruption of expression.

36. A method of improving efficacy of immune cell therapy, comprising the step of disrupting expression of SIT1, BST2, and PD-1 in the immune cells of the immune cell therapy.

37. The method of claim 36, wherein the disrupting of expression in the cells overcomes immunosuppression for the cells in the microenvironment of a tumor.

38. A method of treating an individual for cancer, comprising the step of providing to the individual an effective amount of any one of the compositions of claims 1-23.

39. The method of claim 38, wherein the immune cells are obtained from the cancer of the individual.

40. The method of claim 38 or 39, further defined as:
(a) expanding immune cells obtained from the cancer of the individual;
(b) disrupting expression of SIT1, BST2, and PD-1 in the cells to produce engineered cells; and
(c) administering an effective amount of the engineered cells to the individual.

41. The method of claim 40, further comprising the step of:
(d) modifying the engineered cells to express one or more engineered receptors.

42. The method of claim 41, wherein the engineered receptors are engineered antigen receptors.

43. The method of claim 42, wherein the engineered antigen receptor is a chimeric antigen receptor or an engineered T cell receptor.

44. The method of any one of claims 38-43, further comprising the step of providing to the individual an effective amount of an additional cancer therapy.

45. The method of claim 44, wherein the additional cancer therapy comprises radiation therapy, surgery, chemotherapy, gene therapy, DNA therapy, viral therapy, RNA therapy, immunotherapy, bone marrow transplantation, nanotherapy, monoclonal antibody therapy, hormone therapy, or a combination thereof.
